SEQUENCE LISTING (23 sequences)

SEQ ID NO: 1 - protein, 501 aa, synthetic construct
KWSKAGGSFTMLLLVTSLLLCELPHPAFLLIPQVQLVQSGAEVKKPGESLKISCKGSRHSFTSYWIGWVRQMPGKGLEWMGIIYPGDSDTRYSPSFQGQVTISADKSISTAYLQWSSLKASDTAMYYCARRIAAAGIGNEYFDYWGQGTTVTVSSGGGGSGGGGSGGGGSSYELTQPPSVSVSPGQTASITCSGDKLGDKYVSWYQQKPGQSPVLVIYQDSKRPSGIPERFSGSNSGNTATLTISGTQAMDEADYYCQAWDSSVVFGGGTQLIILAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 123 aa, synthetic construct
QVQLVQSGAEVKKPGESLKISCKGSRHSFTSYWIGWVRQMPGKGLEWMGIIYPGDSDTRYSPSFQGQVTISADKSISTAYLQWSSLKASDTAMYYCARRIAAAGIGNEYFDYWGQGTTVTVSS

SEQ ID NO: 3 - protein, 105 aa, synthetic construct
SYELTQPPSVSVSPGQTASITCSGDKLGDKYVSWYQQKPGQSPVLVIYQDSKRPSGIPERFSGSNSGNTATLTISGTQAMDEADYYCQAWDSSVVFGGGTQLIIL

SEQ ID NO: 4 - protein, 503 aa, synthetic construct
KWSKAGGSFTMLLLVTSLLLCELPHPAFLLIPQVQLQQSGPGLVKPSQTLSLTCAISGDSVSSNSAAWNWIRQSPSRGLEWLGRAYYRSTWYNDYAASVKSRITINPDTSKNQFSLQLNSVTPEDTAVYYCARGVNWNDDAFDIWGQGTMVTVSSGGGGSGGGGSGGGGSAIQMTQSPSSLSASVGDRVTITCRASQSISSYLNWYQQKPGKAPKLLVYAASSLQSGVPSRFSGSGSGTDFTLTISSLQPEDFATYYCQQSYSTPLTFGGGTKVEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 123 aa, synthetic construct
QVQLQQSGPGLVKPSQTLSLTCAISGDSVSSNSAAWNWIRQSPSRGLEWLGRAYYRSTWYNDYAASVKSRITINPDTSKNQFSLQLNSVTPEDTAVYYCARGVNWNDDAFDIWGQGTMVTVSS

SEQ ID NO: 6 - protein, 107 aa, synthetic construct
AIQMTQSPSSLSASVGDRVTITCRASQSISSYLNWYQQKPGKAPKLLVYAASSLQSGVPSRFSGSGSGTDFTLTISSLQPEDFATYYCQQSYSTPLTFGGGTKVEIK

SEQ ID NO: 7 - protein, 15 aa, synthetic construct
GGGGSGGGGSGGGGS

SEQ ID NO: 8 - protein, 72 aa, synthetic construct
AAATTTPAPRPPTPAPTIASQPLSLRPEACRPAAGGAVHTRGLDFACDIYIWAPLAGTCGVLLLSLVITLYC

SEQ ID NO: 9 - protein, 41 aa, synthetic construct
KRGRKKLLYIFKQPFMRPVQTTQEEDGCSCRFPEEEEGGCE

SEQ ID NO: 10 - protein, 113 aa, synthetic construct
LRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 751 aa, synthetic construct
METDTLLLWVLLLWVPGSTGQVQLVQSGAEVKKPGESLKISCKGSRHSFTSYWIGWVRQMPGKGLEWMGIIYPGDSDTRYSPSFQGQVTISADKSISTAYLQWSSLKASDTAMYYCARRIAAAGIGNEYFDYWGQGTTVTVSSGGGGSGGGGSGGGGSSYELTQPPSVSVSPGQTASITCSGDKLGDKYVSWYQQKPGQSPVLVIYQDSKRPSGIPERFSGSNSGNTATLTISGTQAMDEADYYCQAWDSSVVFGGGTQLIILAAAGGGGSGGGGSGGGGSQVQLQQSGPGLVKPSQTLSLTCAISGDSVSSNSAAWNWIRQSPSRGLEWLGRAYYRSTWYNDYAASVKSRITINPDTSKNQFSLQLNSVTPEDTAVYYCARGVNWNDDAFDIWGQGTMVTVSSGGGGSGGGGSGGGGSAIQMTQSPSSLSASVGDRVTITCRASQSISSYLNWYQQKPGKAPKLLVYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLE

SEQ ID NO: 12 - protein, 123 aa, synthetic construct; identical to SEQ ID NO: 2

SEQ ID NO: 13 - protein, 108 aa, synthetic construct
SYELTQPPSVSVSPGQTASITCSGDKLGDKYVSWYQQKPGQSPVLVIYQDSKRPSGIPERFSGSNSGNTATLTISGTQAMDEADYYCQAWDSSVVFGGGTQLIILAAA

SEQ ID NO: 14 - protein, 123 aa, synthetic construct; identical to SEQ ID NO: 5

SEQ ID NO: 15 - protein, 107 aa, synthetic construct; identical to SEQ ID NO: 6

SEQ ID NO: 16 - protein, 751 aa, synthetic construct
METDTLLLWVLLLWVPGSTGQVQLVQSGAEVKKPGESLKISCKGSRHSFTSYWIGWVRQMPGKGLEWMGIIYPGDSDTRYSPSFQGQVTISADKSISTAYLQWSSLKASDTAMYYCARRIAAAGIGNEYFDYWGQGTTVTVSSGGGGSGGGGSGGGGSSYELTQPPSVSVSPGQTASITCSGDKLGDKYVSWYQQKPGQSPVLVIYQDSKRPSGIPERFSGSNSGNTATLTISGTQAMDEADYYCQAWDSSVVFGGGTQLIILAAAEAAAKEAAAKEAAAKQVQLQQSGPGLVKPSQTLSLTCAISGDSVSSNSAAWNWIRQSPSRGLEWLGRAYYRSTWYNDYAASVKSRITINPDTSKNQFSLQLNSVTPEDTAVYYCARGVNWNDDAFDIWGQGTMVTVSSGGGGSGGGGSGGGGSAIQMTQSPSSLSASVGDRVTITCRASQSISSYLNWYQQKPGKAPKLLVYAASSLQSGVPSRFSGSGSGTDFTLTISSLQPEDFATYYCQQSYSTPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLE

SEQ ID NO: 17 - protein, 143 aa, synthetic construct
METDTLLLWVLLLWVPGSTGQVQLVQSGAEVKKPGESLKISCKGSRHSFTSYWIGWVRQMPGKGLEWMGIIYPGDSDTRYSPSFQGQVTISADKSISTAYLQWSSLKASDTAMYYCARRIAAAGIGNEYFDYWGQGTTVTVSS

SEQ ID NO: 18 - protein, 108 aa, synthetic construct; identical to SEQ ID NO: 13

SEQ ID NO: 19 - protein, 123 aa, synthetic construct; identical to SEQ ID NO: 5

SEQ ID NO: 20 - protein, 107 aa, synthetic construct; identical to SEQ ID NO: 6

SEQ ID NO: 21 - protein, 15 aa, synthetic construct
EAAAKEAAAKEAAAK

SEQ ID NO: 22 - protein, 754 aa, synthetic construct
METDTLLLWVLLLWVPGSTGQVQLVQSGAEVKKPGESLKISCKGSRHSFTSYWIGWVRQMPGKGLEWMGIIYPGDSDTRYSPSFQGQVTISADKSISTAYLQWSSLKASDTAMYYCARRIAAAGIGNEYFDYWGQGTTVTVSSGGGGSGGGGSGGGGSSYELTQPPSVSVSPGQTASITCSGDKLGDKYVSWYQQKPGQSPVLVIYQDSKRPSGIPERFSGSNSGNTATLTISGTQAMDEADYYCQAWDSSVVFGGGTQLIILAAAGGGGSGGGGSGGGGSQVQLQQSGPGLVKPSQTLSLTCAISGDSVSSNSAAWNWIRQSPSRGLEWLGRAYYRSTWYNDYAASVKSRITINPDTSKNQFSLQLNSVTPEDTAVYYCARGVNWNDDAFDIWGQGTMVTVSSGGGGSGGGGSGGGGSAIQMTQSPSSLSASVGDRVTITCRASQSISSYLNWYQQKPGKAPKLLVYAASSLQSGVPSRFSGSGSGTDFTLTISSLQPEDFATYYCQQSYSTPLTFGGGTKVEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLE

SEQ ID NO: 23 - protein, 754 aa, synthetic construct
METDTLLLWVLLLWVPGSTGQVQLVQSGAEVKKPGESLKISCKGSRHSFTSYWIGWVRQMPGKGLEWMGIIYPGDSDTRYSPSFQGQVTISADKSISTAYLQWSSLKASDTAMYYCARRIAAAGIGNEYFDYWGQGTTVTVSSGGGGSGGGGSGGGGSSYELTQPPSVSVSPGQTASITCSGDKLGDKYVSWYQQKPGQSPVLVIYQDSKRPSGIPERFSGSNSGNTATLTISGTQAMDEADYYCQAWDSSVVFGGGTQLIILAAAEAAAKEAAAKEAAAKQVQLQQSGPGLVKPSQTLSLTCAISGDSVSSNSAAWNWIRQSPSRGLEWLGRAYYRSTWYNDYAASVKSRITINPDTSKNQFSLQLNSVTPEDTAVYYCARGVNWNDDAFDIWGQGTMVTVSSGGGGSGGGGSGGGGSAIQMTQSPSSLSASVGDRVTITCRASQSISSYLNWYQQKPGKAPKLLVYAASSLQSGVPSRFSGSGSGTDFTLTISSLQPEDFATYYCQQSYSTPLTFGGGTKVEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLE

(19) United States
(12) Patent Application Publication
(10) Pub. No.: US 2025/0121007 A1
(43) Pub. Date: Apr. 17, 2025

(54) COMPOSITIONS FOR THE TREATMENT OF CANCER

(71) Applicant: GenCART, Inc., Pittsburgh, PA (US)

(72) Inventors: Dimiter S. DIMITROV, Pittsburgh, PA (US); M. Mounzer E. AGHA, Pittsburgh, PA (US)

(73) Assignee: GenCART, Inc., Pittsburgh, PA (US)

(21) Appl. No.: 18/914,989

(22) Filed: Oct. 14, 2024

Related U.S. Application Data
(60) Provisional application No. 63/543,994, filed on Oct. 13, 2023.

(51) Int. Cl. A61K 35/17; A61K 39/00; C07K 16/28

(52) U.S. Cl. CPC A61K 35/17; A61K 40/11; A61K 40/15; A61K 40/31; A61K 40/4211; A61K 40/4212; C07K 16/2803; A61K 2239/13; A61K 2239/21

(57) ABSTRACT

The present invention provides nucleic acid sequences encoding chimeric antigen receptors (CAR), wherein the CAR comprises an antigen binding domain, an extracellular domain, a transmembrane domain, intracellular stimulatory and co-stimulatory domains. The present invention also provides chimeric DNA comprising a first DNA segment encoding a single-chain variable fragment (scFv) of an antibody comprising a heavy chain variable region (VH) linked to a light chain variable region (VL) by a flexible linker. The present invention also provides a chimeric DNA wherein the endogenous protein is expressed on the surface of a lymphocyte or macrophage and triggers the activation of the lymphocyte or macrophage. The present invention also provides an expression vector comprising a chimeric DNA. The present invention also provides a lymphocyte or macrophage transformed with an expression vector or chimeric DNA wherein expression of the expression vector or chimeric DNA endows the lymphocyte or macrophage with antibody specificity.

### CROSS-REFERENCE TO RELATED APPLICATIONS

This application claims priority to U.S. Provisional Application No. 63/543,994 filed on Oct. 13, 2023, which is incorporated herein by reference in its entirety.

### SEQUENCE LISTING

The instant application contains a Sequence Listing which has been submitted electronically in extensible Markup Language (XML) format and is hereby incorporated by reference in its entirety. The XML copy, created on Oct. 14, 2024, is named “441368-000101_Sequence_Listing.xml” and is 26,300 bytes in size.

### BACKGROUND OF THE INVENTION

Great progress has been made by treating hematological cancers with T-cell therapies targeting CD19. However, relapse rates remain high due to tumor heterogeneity, loss of CD19 and CD19 downregulation. Targeting additional tumor-associated antigens is an important therapeutic strategy to address these tumor escape mechanisms. One such antigen is CD22, restricted to B lymphocytes and expressed or overexpressed in various B lymphocyte malignancies and autoimmune disorders.

### SUMMARY OF THE INVENTION

The present invention provides nucleic acid sequences encoding chimeric antigen receptors (CAR), wherein the CAR comprises an antigen binding domain, an extracellular domain, a transmembrane domain and intracellular stimulatory and co-stimulatory domains (SEQ ID NO: 1, SEQ ID NO: 4, SEQ ID NO: 11 and SEQ ID NO: 16).

In one embodiment, the invention provides a chimeric DNA comprising: a first DNA segment encoding a single-chain variable fragment (scFv) of an antibody comprising a heavy chain variable region (VH) (SEQ ID NO: 2) linked to a light chain variable region (VL) (SEQ ID NO: 3) by a flexible linker (SEQ ID NO: 7) or a first DNA segment encoding a single-chain variable fragment of an antibody comprising a VH (SEQ ID NO: 5) linked to a VL (SEQ ID NO: 6) by a flexible linker (SEQ ID NO: 7), or a first DNA segment encoding a single-chain variable domain comprising a VH (SEQ ID NO: 12) linked by a flexible linker (SEQ ID NO: 7) to a VL (SEQ ID NO: 13) linked by a flexible linker (SEQ ID NO: 7) to a second DNA segment encoding a single-chain variable domain comprising a VH (SEQ ID NO: 14) linked to a VL (SEQ ID NO: 15) by a flexible linker (SEQ ID NO: 7), or a first DNA segment encoding a single-chain variable domain comprising a VH (SEQ ID NO: 17) linked by a flexible linker (SEQ ID NO: 7) to a VL (SEQ ID NO: 18) linked by a rigid linker (SEQ ID NO: 21) to a second DNA segment encoding a single-chain variable domain comprising a VH (SEQ ID NO: 19) linked to a VL (SEQ ID NO: 20) by a flexible linker (SEQ ID NO: 7) and a second DNA encoding partially or entirely an extracellular domain comprising at least a portion of the CD8 alpha extracellular domain (SEQ ID NO: 8), a transmembrane domain and intracellular domain of an endogenous protein and optionally stimulatory (SEQ ID NO: 10) and co-stimulatory (SEQ ID NO: 9) domains of an endogenous protein.

In one aspect, the invention provides a chimeric DNA wherein the endogenous protein is expressed on the surface of a lymphocyte or macrophage and triggers the activation of the lymphocyte or macrophage.

In one aspect, the invention provides a chimeric DNA wherein the chimeric DNA, upon transfection or transduction to the lymphocyte or macrophage, expresses both the single-chain variable domain and the domains of the endogenous protein in one single, continuous chain (SEQ ID NO: 1, SEQ ID NO: 4, SEQ ID NO: 11 and SEQ ID NO: 16)) on the surface of the transfected or transduced lymphocytes or macrophages such that the transfected or transduced lymphocyte or macrophage is triggered to activate and/or proliferate and have major histocompatibility complex (MHC) non-restricted antibody-type specificity when the expressed single chain variable domain binds to its antigen.

In one aspect, the invention provides a chimeric DNA wherein the chimeric DNA, upon transfection or transduction to the lymphocyte or macrophage, expresses both the single-chain variable domain and the domains of the endogenous protein in one single, continuous chain (SEQ ID NO: 1, SEQ ID NO: 4, SEQ ID NO: 22 and SEQ ID NO: 23)) on the surface of the transfected or transduced lymphocytes or macrophages such that the transfected or transduced lymphocyte or macrophage is triggered to activate and/or proliferate and have major histocompatibility complex (MHC) non-restricted antibody-type specificity when the expressed single chain variable domain binds to its antigen.

In another aspect, the invention provides a chimeric DNA wherein the second DNA segment further comprises partially or entirely the extracellular domain of the endogenous protein.

In one aspect, the invention provides a chimeric DNA wherein the second DNA segment further comprises partially or entirely the transmembrane domain of the endogenous protein.

In another aspect, the invention provides a chimeric DNA wherein the second DNA segment further comprises partially or entirely the co-stimulatory domain of the endogenous protein.

In one embodiment, the invention provides a chimeric DNA wherein the second DNA segment comprises either entirely or partially domains of the CD28 family of receptors.

In one aspect, the invention provides a chimeric DNA wherein the second DNA segment comprises either entirely or partially domains of CD28 or ICOS.

In one embodiment, the invention provides a chimeric DNA wherein the second DNA segment comprises entirely or partially domains of the TNFR superfamily.

In one aspect, the invention provides a chimeric DNA wherein the second DNA segment comprises entirely or partially domains of CD137, CD30, CD40, OX40, CD27, CD226, GITR, RANK, TACI, BCMA and BAFF-R.

In one embodiment, the invention provides a chimeric DNA wherein the second DNA segment further comprises partially or entirely a lymphocyte receptor chain.

In one aspect, the invention provides a chimeric DNA wherein the second DNA segment comprises either entirely or partially a chain of the T-cell receptor (TCR).

In another aspect, the invention provides a chimeric DNA wherein the second DNA segment encodes the & chain of the TCR.

In one embodiment, the invention provides an expression vector comprising a chimeric DNA.

In one embodiment, the invention provides a lymphocyte or macrophage, isolated from an individual, transformed with an expression vector wherein expression of the chimeric DNA endows the lymphocyte or macrophage with antibody specificity.

In one aspect, the invention provides a lymphocyte or macrophage, isolated from an individual, transformed with a chimeric DNA wherein expression of the chimeric DNA endows the lymphocyte or macrophage with antibody specificity.

In another aspect, the invention provides a macrophage or a lymphocyte selected from the group consisting of, but not limited to the following types of lymphocytes, a natural killer cell, a natural killer T-cell, a lymphokine activated killer cell, a cytotoxic T-cell, a helper T-cell, a gamma delta T-cell and a subtype thereof.

In one embodiment, the invention provides a chimeric DNA wherein the first DNA segment encodes the single-chain variable domain of an antibody targeting a protein expressed or overexpressed on tumor cells.

In one aspect, the invention provides a chimeric DNA wherein the first DNA segment encodes the single-chain variable domain of an antibody targeting tumor cells either expressing or overexpressing CD19 or CD22, or a combination thereof.

In one embodiment, the invention provides a chimeric DNA comprising SEQ ID NO: 1.

In another embodiment, the invention provides a chimeric DNA comprising SEQ ID NO: 4.

In another embodiment, the invention provides a chimeric DNA comprising SEQ ID NO: 11.

In another embodiment, the invention provides a chimeric DNA comprising SEQ ID NO: 16.

In another embodiment, the invention provides a chimeric DNA comprising SEQ ID NO: 22.

In another embodiment, the invention provides a chimeric DNA comprising SEQ ID NO: 23.

### DETAILED DESCRIPTION

The invention relates to compositions and methods for treating hematologic cancers, including but not limited to B cell malignancies, and autoimmune disorders, including but not limited to lupus, multiple sclerosis, and rheumatoid arthritis. The present invention relates to a strategy of adoptive cell transfer of T-cells, natural killer cells (NK), natural killer T-cells, lymphokine activated killer cells and macrophages genetically engineered to express a chimeric antigen receptor (CAR) that recognize antigens expressed on tumor cells. CARs are comprised of an antigen binding region, for example, a single-chain variable fragment (scFv) derived from an antigen-specific monoclonal antibody and a T-cell activation molecule, such as the CD3 ζ-chain of the T-cell receptor (TCR).

The present invention relates generally to the use of genetically modified T-cells, natural killer cells, natural killer T-cells, lymphokine activated killer cells and macrophages stably expressing a desired CAR on the T-cell surface. In some instances, the T-cell, natural killer cell, natural killer T-cell, lymphokine activated killer cell and macrophage is genetically modified to stably express a CAR that combines an antigen recognition domain of an antibody or antibody fragment with an intracellular stimulatory domain of CD3-zeta, a co-stimulatory domain, or a combination thereof.

In one embodiment, a chimeric DNA comprises a first DNA segment encoding an scFv of an antibody comprising a VH (SEQ ID NO: 2) linked to a VL (SEQ ID NO: 3) by a flexible linker (SEQ ID NO: 7), a first DNA segment encoding a scFv of an antibody comprising a VH (SEQ ID NO: 5) linked to a VL (SEQ ID NO: 6) by a flexible linker (SEQ ID NO: 7), or a first DNA segment encoding a single-chain variable domain comprising a VH (SEQ ID NO: 12) linked by a flexible linker (SEQ ID NO: 7) to a VL (SEQ ID NO: 13) linked by a flexible linker (SEQ ID NO: 7) to a second DNA segment encoding a single-chain variable domain comprising a VH (SEQ ID NO: 14) linked to a VL (SEQ ID NO: 15) by a flexible linker (SEQ ID NO: 7), or a first DNA segment encoding a single-chain variable domain comprising a VH (SEQ ID NO: 17) linked by a flexible linker (SEQ ID NO: 7) to a VL (SEQ ID NO: 18) linked by a rigid linker (SEQ ID NO: 21) to a second DNA segment encoding a single-chain variable domain comprising a VH (SEQ ID NO: 19) linked to a VL (SEQ ID NO: 20) by a flexible linker (SEQ ID NO: 7) and a second DNA encoding partially or entirely an extracellular domain comprising at least a portion of the CD8 alpha extracellular domain (SEQ ID NO: 8), a transmembrane domain and intracellular domain of an endogenous protein and optionally stimulatory (SEQ ID NO: 10) and co-stimulatory (SEQ ID NO: 9) domains of an endogenous protein.

In some embodiments, an antibody comprises a VH and a VL. In some embodiments, an antibody comprises at least one heavy chain (HC) comprising a VH and at least a portion of a heavy chain constant region, and at least one light chain (LC) comprising a VL and at least a portion of a light chain constant region. In some embodiments, an antibody comprises two heavy chains, wherein each heavy chain comprises a VH and at least a portion of a heavy chain constant region, and two light chains, wherein each light chain comprises a VL and at least a portion of a light chain constant region. The VH and VL are generally connected together using a flexible glycine-serine linker, and then attached to a transmembrane domain by an extracellular spacer, in some embodiments, comprising the entire or a portion of the extracellular domain of CD8 alpha, to extend the scFv away from the T-cell, natural killer cell, natural killer T-cell, lymphokine activated killer cell and macrophage surface so that it can interact with tumor antigens.

The present invention provides a CAR or CARs comprising extracellular and intracellular domains. The extracellular domain comprises a target-specific binding element and a spacer domain to separate the scFv from the cell surface. The intracellular domain comprises a co-stimulatory signaling region and a CD3 ζ-chain portion. The co-stimulatory signaling region refers to a portion of the CAR comprising the intracellular domain of a costimulatory molecule. Costimulatory molecules are cell surface molecules other than antigen receptors or their ligands that are required for an efficient response of lymphocytes or macrophages to antigen.

With respect to the intracellular domain, the CAR of the invention can be designed to comprise the CD28 and/or 4-1BB signaling domain by itself of be combined with any other desired intracellular domains useful in the context of the CAR of the present invention. In one embodiment, the intracellular domain of the CAR can be designed to further comprise the signaling domain of CD3 ζ. For example, the intracellular domain of the CAR can include but is not limited to CD3 ζ, 4-1BB and CD28 signaling modules and combinations thereof. The CAR of the present invention may also comprise signaling domains from ICOS, CD137, CD30, CD40, OX40, CD27, CD226, GITR, RANK, TACI, BCMA and BAFF-R. Accordingly, the invention provides CAR-T, NK-CAR and macrophage-CAR cells and methods of their use in adoptive cell therapy.

In one embodiment, the CAR-T, NK-CAR and macrophage-CAR cells of the invention can be generated by introducing a lentiviral, gammaretroviral, adenoviral or adeno-associated viral vector comprising a desired CAR, for example a CAR comprising anti-CD19 or anti-CD22, CD8 alpha extracellular domain, transmembrane domain, and CD28, 4-1BB and CD3 ζ signaling domains, into the cells.

The present invention is also directed to methods of treating cancer in a subject, comprising administering to the subject a therapeutically effective amount of transformed lymphocytes or macrophages according with the instant disclosure. In some embodiments, cancer is a B cell malignancy. In some embodiments, the B cell malignancy is selected from the group consisting of acute lymphoblastic leukemia, chronic lymphocytic leukemia, non-Hodgkin lymphoma, and multiple myeloma.

The present invention is also directed to methods of treating an autoimmune disorder in a subject, comprising administering to the subject a therapeutically effective amount of transformed lymphocytes or macrophages in accordance with the instant disclosure. In some embodiments, the autoimmune disorder is selected from the group consisting of lupus, multiple sclerosis, and rheumatoid arthritis.

The present invention is also directed to methods of depleting B cells in a subject, comprising administering to the subject an effective amount of transformed lymphocytes or macrophages in accordance with the instant disclosure, wherein the transformed lymphocytes or macrophages express a chimeric antigen receptor targeting CD19 or CD22.

The present invention is also directed to methods of producing a population of transformed lymphocytes or macrophages, comprising isolating lymphocytes or macrophages from a subject; transducing the isolated lymphocytes or macrophages with the expression vectors of the instant disclosure; and culturing the transduced lymphocytes or macrophages under conditions that allow for expression of the chimeric DNA. Some embodiments further comprise expanding the cultured transformed lymphocytes or macrophages to produce a therapeutic dose.

The term “antibody” in the broadest sense, refers to glycoproteins having the same structural characteristics (i.e., molecules that contain an antigen binding site that immunospecifically binds an antigen) and encompass various antibody structures, including natural or artificial, mono- or polyvalent antibodies including but not limited to monoclonal antibodies (including chimeric monoclonal antibodies, humanized monoclonal antibodies, and human monoclonal antibodies, particularly humanized monoclonal antibodies), polyclonal antibodies, single chain antibodies, antibody fragments, and multispecific antibodies (e.g., bispecific antibodies). While antibodies exhibit binding specificity to a specific antigen, immunoglobulins include both antibodies and other antibody-like molecules which lack antigen specificity. “Antibody,” encompasses any polypeptide comprising an antigen-binding site regardless of the source, species of origin, method of production, and characteristics. The term “antibody” may also broadly refer to a molecule comprising complementarity determining region (CDR) 1, CDR2, and CDR3 of a heavy chain and CDR1, CDR2, and CDR3 of a light chain, wherein the molecule is capable of binding to an antigen.

The term “antibody heavy chain variable region” in the broadest sense, refers to at a minimum, a region comprising heavy chain CDR1 (CDR-H1), framework 2 (HFR2), CDR2 (CDR-H2), FR3 (HFR3), and CDR3 (CDR-H3). In some embodiments, a heavy chain variable region also comprises at least a portion (e.g., the whole) of an FR1 (HFR1), which is N-terminal to CDR-H1, and/or at least a portion (e.g., the whole) of an FR4 (HFR4), which is C-terminal to CDR-H3.

The term “antibody light chain variable region” in the broadest sense, refers to at a minimum, a region comprising light chain CDR1 (CDR-L1), framework (FR) 2 (LFR2), CDR2 (CDR-L2), FR3 (LFR3), and CDR3 (CDR-L3). In some embodiments, a light chain variable region also comprises at least a portion (e.g., the whole) of an FR1 (LFR1), which is N-terminal to CDR-L1, and/or at least a portion (e.g., the whole) of an FR4 (LFR4), which is C-terminal to CDR-L3.

The term “flexible linker” in the broadest sense, refers to a polypeptide sequence comprising glycine and serine that connect together the VH and VL of a CAR scFv. These polypeptide sequences generally comprise of single or repeats of glycine-serine-glycine or glycine-glycine-glycine-glycine-serine. The combination of flexible and hydrophilic residues in these linkers prevents the formation of secondary structures and reduces the likelihood that the linkers will interfere with the folding and function of the protein domains. Specifically, they are meant to connect a VH with a VL without interfering with the ability of the combination to bind its target antigen. The current invention utilizes a (G₄)₃ flexible linker (SEQ ID NO: 7).

The term “rigid linker” in the broadest sense, refers to a polypeptide sequence comprising amino acids that adopt helical conformations to maintain a fixed distance between adjoining components to maintain their independent functions. The current invention utilizes a (EA₃K)₃ rigid linker (SEQ ID NO: 21) to separate two scFvs so as to maintain freedom for each scFv to freely bind their distinct target antigen.

The terms “extracellular domain” or “CD8 alpha extracellular domain” in the broadest sense, refer to a spacer domain between the CAR scFv and the transmembrane domain. As used herein, the term “extracellular domain” generally means any oligo- or polypeptide that functions to link the transmembrane domain to the CAR scFv. The current invention utilizes a portion of or the entire extracellular domain of CD8 alpha.

The term “transmembrane domain” in the broadest sense refers to a plasma membrane-spanning protein domain. Transmembrane domains (TMD) may consist of one or several alpha-helicies or a transmembrane beta barrel. Because the interior of the plasma membrane is hydrophobic, the amino acid residues in TMDs are often hydrophobic. In the current invention, the function of TMD is for anchoring the CAR molecule to the plasma membrane and to facilitate signal transduction from the scFv binding its target, passing it through the membrane resulting in activation of the intracellular stimulatory and co-stimulatory domains.

The term “intracellular domain” in the broadest sense refers to the portion of the CAR molecule that extends from the junction of the transmembrane domain and interior of the plasma membrane, thereby residing within the cytoplasm of the cell. Normal T-cell activation relies on the phosphorylation of stimulatory domains comprising of three immunoreceptor tyrosine-based activation motifs (ITAM) present in the cytoplasmic domain of CD3 ζ. Since T-cells also require co-stimulatory molecules in addition to CD3 signaling in order to persist after activation, for this reason the cytoplasmic domains of CAR molecules typically also include one or more chimeric signaling domains from co-stimulatory proteins.

The term “stimulatory domain” in the broadest sense, refers to the CD3 & chain of the TCR, that when the TCR binds its cognate peptide in the context of major histocompatibility complex (MHC), elicits a signal transduction event including, but not limited to, activation, initiation of an immune response, proliferation and the like.

The term “co-stimulatory domain” in the broadest sense, refers to the intracellular portion of a co-stimulatory cell surface receptor that when bound by its cognate co-stimulatory ligand elicits a signal, that in addition to the primary signal provided by the TCR & chain upon receptor binding its cognate peptide in the context of MHC, mediates a cellular response, including but not limited to proliferation, activation, differentiation and the like. Examples of co-stimulatory cell surface receptors are CD28, ICOS, CD137, CD30, CD40, OX40, CD27, CD226, GITR, RANK, TACI, BCMA and BAFF-R.

The term “antigen” in the broadest sense, refers to a molecule that provokes an immune response. This immune response may involve either antibody production, or the activation of specific immunologically-competent cells, or both. The skilled artisan will understand that any macromolecule, including virtually all proteins or peptides, can serve as an antigen. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response.

The term “endogenous” in the broadest sense, refers to any material from or produced inside an organism, cell, tissue or system.

The term “lymphocyte” in the broadest sense, refers to a type of white blood cell (leukocyte) in the immune system of most vertebrates. Lymphocytes include T-cells, B cells, natural killer (NK) cells, and natural killer T-cells (NKT).

The terms “transfection” or “transduction” in the broadest sense, refer to a process by which exogenous nucleic acid is transferred or introduced into a host cell. A transfected or transduced cell is one which has been transfected or transduced with exogenous nucleic acid. The cell includes the primary subject and its progeny.

The term “expression vector” in the broadest sense, refers to a vector comprising a recombinant polynucleotide comprising expression control sequences operatively linked to a nucleotide sequence to be expressed. An expression vector comprises sufficient cis-acting elements for expression; other elements for expression can be provided by the host cell or in an in vitro expression system. Expression vectors include all those known in the art, including cosmids, plasmids (e.g., naked or encapsulated in liposomes) and viruses (e.g., lentiviruses, gammaretroviruses, adenoviruses and adeno-associated viruses) that incorporate the recombinant polynucleotide.

The term “polynucleotide” in the broadest sense, refers to a compound comprised of no fewer than two amino acid residues covalently linked by peptide bonds.

The term “MHC non-restricted antibody-type specificity” in the broadest sense, refers to the ability of a monoclonal antibody or antibody fragment to recognize its target antigen in the absence of presentation by an MHC molecule. TCRs recognize short linear peptide antigens only if coupled with an MHC molecule. Specifically, the ligands of the TCR are specific peptide-MHC complexes. There are six different MHC class I molecules and eight different MHC class II molecules. Any one particular peptide antigen can only be presented to a TCR by one particular MHC class I or class II molecule. Humans contain varying combinations of these MHCs. If two people do not share the same MHC combinations, a particular TCR that recognizes a peptide that is restricted to a particular MHC from one individual may not recognize that same peptide antigen if found or experimentally introduced to another individual. Replacing the TCR with a CAR scFv as the antigen binding region on a T-cell eliminates MHC restriction and greatly broadens the available antigens targeted by CARs.

The term “CD28 family” in the broadest sense, refers to a group of regulatory cell surface receptors found on the cell surface of immune cells. Two members, CD28 and ICOS, are positive regulators of T-cell function while another three, BTLA, CTLA-4 and PD-1 act as inhibitors. CD28 receptors are integral regulators of T-cell development and proliferation by enhancing signals from the TCR in order to modulate the immune response.

The term “TNFR superfamily” in the broadest sense, refers to a protein superfamily of cytokine receptors characterized by the ability to bind tumor necrosis factors (TNF) via a structurally homologous ectodomain defined by a cysteine-rich domain. In their active form, the majority of TNF receptors form trimeric complexes in the plasma membrane. Most TNF receptors require specific adaptor proteins to elicit activation of downstream signaling cascades that regulate immune responses across various immune cell populations. Examples of TNFR superfamily members include, but are not limited to, CD137, CD30, CD40, OX40, CD27, CD226, GITR, RANK, TACI, BCMA and BAFF-R.

The term “T-cell receptor” in the broadest sense, refers to a protein complex found on the surface of T-cells that is responsible for recognizing fragments of antigen as peptides bound to MHC molecules. The TCR is composed of two different protein chains. In humans, in 95% of T cells the TCR consists of an alpha (α) chain and a beta (β) chain, whereas in 5% of T cells the TCR consists of gamma and delta (γ/δ) chains. Each locus can produce a variety of polypeptides with constant and variable regions. When the TCR engages with antigenic peptide and MHC (peptide/MHC), the T-cell is activated through signal transduction, that is, a series of biochemical events mediated by associated enzymes, co-receptors, specialized adaptor molecules, and activated or released transcription factors. Based on the initial receptor triggering mechanism, the TCR belongs to the family of non-catalytic tyrosine-phosphorylated receptors (NTRs).

The term “ζ chain of the T-cell receptor” in the broadest sense, refers to the T-cell surface glycoprotein CD3 zeta chain, also referred to as CD247. The CD3 ζ, together with the TCR α/β and γ/δ chains, form the TCR CD3 complex. The zeta chain plays an important role in coupling antigen recognition to several intracellular signal transduction pathways by way of its three immunoreceptor tyrosine-based activation motifs (ITAMs).

The term “CD19” in the broadest sense, refers to the B cell antigen, also known as CD19 molecule, which is a transmembrane protein that in humans is encoded by the gene CD19. In humans, CD19 is expressed in all B lineage cells. CD19 plays two major roles in human B cells: it acts as an adapter protein to recruit intracellular signaling proteins to the plasma membrane and it also complexes with CD21 to lower the threshold for B cell receptor signaling pathways. Due to its presence on all B cells, it is a biomarker for B cell development, lymphoma diagnosis and can be utilized as a target for leukemia and lymphoma therapies.

The term “CD22” in the broadest sense, refers to a molecule belonging to the SIGLEC family of lectins. It is found on the surface of mature B cells and to a lesser extent immature B cells. CD22 functions as an inhibitory receptor for the B cell receptor by preventing overactivation of B cells. Due to its expression on B cells, CD22 serves as a target for leukemia and lymphoma therapies.

Sequences:
Underlined: CDRs
>Full length open reading frame
(SEQ ID NO: 1)
KWSKAGGSFTMLLLVTSLLLCELPHPAFLLIPQVQLVQSGAEVKKPGESLKISCKGSRHS
FTSYWIGWVRQMPGKGLEWMGIIYPGDSDTRYSPSFQGQVTISADKSISTAYLQWSSLKA
SDTAMYYCARRIAAAGIGNEYFDYWGQGTTVTVSSGGGGSGGGGSGGGGSSYELTQPPSV
SVSPGQTASITCSGDKLGDKYVSWYQQKPGQSPVLVIYqdskrpsGIPERFSGSNSGNTA
TLTISGTQAMDEADYYCQAWDSSVVFGGGTQLIILAAATTTPAPRPPTPAPTIASQPLSL
RPEACRPAAGGAVHTRGLDFACDIYIWAPLAGTCGVLLLSLVITLYCKRGRKKLLYIFKQ
PFMRPVQTTQEEDGCSCRFPEEEEGGCELRVKFSRSADAPAYKQGQNQLYNELNLGRREE
YDVLDKRRGRDPEMGGKPRRKNPQEGLYNELQKDKMAEAYSEIGMKGERRRGKGHDGLYQ
GLSTATKDTYDALHMQALPPR
>scFv VH
(SEQ ID NO: 2)
QVQLVQSGAEVKKPGESLKISCKGSRHSFTSYWIGWVRQMPGKGLEWMGIIYPGDSDTRY
SPSFQGQVTISADKSISTAYLQWSSLKASDTAMYYCARRIAAAGIGNEYFDYWGQGTTVT
VSS
>scFv VL
(SEQ ID NO: 3)
SYELTQPPSVSVSPGQTASITCSGDKLGDKYVSWYQQKPGQSPVLVIYQDSKRPSGIPER
FSGSNSGNTATLTISGTQAMDEADYYCQAWDSSVVFGGGTQLIIL
>Full length open reading frame
(SEQ ID NO: 4)
KWSKAGGSFTMLLLVTSLLLCELPHPAFLLIPQVQLQQSGPGLVKPSQTLSLTCAISGDS
VSSNSAAWNWIRQSPSRGLEWLGRAYYRSTWYNDYAASVKSRITINPDTSKNQFSLQLNS
VTPEDTAVYYCARGVNWNDDAFDIWGQGTMVTVSSGGGGSGGGGSGGGGSAIQMTQSPSS
LSASVGDRVTITCRASQSISSYLNWYQQKPGKAPKLLVYAASSLQSGVPSRFSGSGSGTD
FTLTISSLOPEDFATYYCQQSYSTPLTFGGGTKVEIKAAATTTPAPRPPTPAPTIASQPL
SLRPEACRPAAGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGL
YQGLSTATKDTYDALHMQALPPR
>scFv VH
(SEQ ID NO: 5)
QVQLQQSGPGLVKPSQTLSLTCAISGDSVSSNSAAWNWIRQSPSRGLEWLGRAYYRSTWY
NDYAASVKSRITINPDTSKNQFSLQLNSVTPEDTAVYYCARGVNWNDDAFDIWGQGTMVT
VSS
>scFv VL
(SEQ ID NO: 6)
AIQMTQSPSSLSASVGDRVTITCRASQSISSYLNWYQQKPGKAPKLLVYAASSLQSGVPS
RFSGSGSGTDFTLTISSLQPEDFATYYCQQSYSTPLTFGGGTKVEIK
>(G₄S)₃ flexible linker
(SEQ ID NO: 7)
GGGGSGGGGSGGGGS
>CD8 alpha
(SEQ ID NO: 8)
AAATTTPAPRPPTPAPTIASQPLSLRPEACRPAAGGAVHTRGLDFACDIYIWAPLAGTCG
VLLLSLVITLYC
>TNFRSF9 (4-1BB)
(SEQ ID NO: 9)
KRGRKKLLYIFKQPFMRPVQTTQEEDGCSCRFPEEEEGGCE
>CD3 ζ chain
(SEQ ID NO: 10)
LRVKFSRSADAPAYKQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
>Full length open reading frame containing
(G₄S)₃ flexible linker
(SEQ ID NO: 11)
METDTLLLWVLLLWVPGSTGQVQLVQSGAEVKKPGESLKISCKGSRHSFTSYWIGWVRQM
PGKGLEWMGIIYPGDSDTRYSPSFQGQVTISADKSISTAYLQWSSLKASDTAMYYCARRI
AAAGIGNEYFDYWGQGTTVTVSSGGGGSGGGGSGGGGSSYELTQPPSVSVSPGQTASITC
SGDKLGDKYVSWYQQKPGQSPVLVIYQDSKRPSGIPERFSGSNSGNTATLTISGTQAMDE
ADYYCQAWDSSVVFGGGTQLIILAAAGGGGSGGGGSGGGGSQVQLQQSGPGLVKPSQTLS
LTCAISGDSVSSNSAAWNWIRQSPSRGLEWLGRAYYRSTWYNDYAASVKSRITINPDTSK
NQFSLQLNSVTPEDTAVYYCARGVNWNDDAFDIWGQGTMVTVSSGGGGSGGGGSGGGGSA
IQMTQSPSSLSASVGDRVTITCRASQSISSYLNWYQQKPGKAPKLLVYAASSLQSGVPSR
FSGSGSGTDFTLTISSLOPEDFATYYCQQSYSTPLTFGGGTKVEIKTTTPAPRPPTPAPT
IASQPLSLRPEACRPAAGGAVHTRGLDFACDIYIWAPLAGTCGVLLLSLVITLYCKRGRK
KLLYIFKQPFMRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSEIGMKGERRRG
KGHDGLYQGLSTATKDTYDALHMQALPPRLE
>scFv VH
(SEQ ID NO: 12)
QVQLVQSGAEVKKPGESLKISCKGSRHSFTSYWIGWVRQMPGKGLEWMGIIYPGDSDTRY
SPSFQGQVTISADKSISTAYLQWSSLKASDTAMYYCARRIAAAGIGNEYFDYWGQGTTVT
VSS
>scFv VL
(SEQ ID NO: 13)
SYELTQPPSVSVSPGQTASITCSGDKLGDKYVSWYQQKPGQSPVLVIYQDSKRPSGIPER
FSGSNSGNTATLTISGTQAMDEADYYCQAWDSSVVFGGGTQLIILAAA
>scFv VH
(SEQ ID NO: 14)
QVQLQQSGPGLVKPSQTLSLTCAISGDSVSSNSAAWNWIRQSPSRGLEWLGRAYYRSTWY
NDYAASVKSRITINPDTSKNQFSLQLNSVTPEDTAVYYCARGVNWNDDAFDIWGQGTMVT
VSS
>scFv VL
(SEQ ID NO: 15)
AIQMTQSPSSLSASVGDRVTITCRASQSISSYLNWYQQKPGKAPKLLVYAASSLQSGVPS
RFSGSGSGTDFTLTISSLQPEDFATYYCQQSYSTPLTFGGGTKVEIK
>Full length open reading frame containing
(EA₃K)₃ rigid linker
(SEQ ID NO: 16)
METDTLLLWVLLLWVPGSTGQVQLVQSGAEVKKPGESLKISCKGSRHSFTSYWIGWVRQM
PGKGLEWMGIIYPGDSDTRYSPSFQGQVTISADKSISTAYLQWSSLKASDTAMYYCARRI
AAAGIGNEYFDYWGQGTTVTVSSGGGGSGGGGSGGGGSSYELTQPPSVSVSPGQTASITC
SGDKLGDKYVSWYQQKPGQSPVLVIYQDSKRPSGIPERFSGSNSGNTATLTISGTQAMDE
ADYYCQAWDSSVVFGGGTQLIILAAAEAAAKEAAAKEAAAKQVQLQQSGPGLVKPSQTLS
LTCAISGDSVSSNSAAWNWIRQSPSRGLEWLGRAYYRSTWYNDYAASVKSRITINPDTSK
NQFSLQLNSVTPEDTAVYYCARGVNWNDDAFDIWGQGTMVTVSSGGGGSGGGGSGGGGSA
IQMTQSPSSLSASVGDRVTITCRASQSISSYLNWYQQKPGKAPKLLVYAASSLQSGVPSR
FSGSGSGTDFTLTISSLQPEDFATYYCQQSYSTPLTFGGGTKVEIKTTTPAPRPPTPAPT
IASQPLSLRPEACRPAAGGAVHTRGLDFACDIYIWAPLAGTCGVLLLSLVITLYCKRGRK
KLLYIFKQPFMRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSEIGMKGERRRG
KGHDGLYQGLSTATKDTYDALHMQALPPRLE
>scFv VH
(SEQ ID NO: 17)
METDTLLLWVLLLWVPGSTGQVQLVQSGAEVKKPGESLKISCKGSRHSFTSYWIGWVRQM
PGKGLEWMGIIYPGDSDTRYSPSFQGQVTISADKSISTAYLQWSSLKASDTAMYYCARRI
AAAGIGNEYFDYWGQGTTVTVSS
>scFv VL
(SEQ ID NO: 18)
SYELTQPPSVSVSPGQTASITCSGDKLGDKYVSWYQQKPGQSPVLVIYQDSKRPSGIPER
FSGSNSGNTATLTISGTQAMDEADYYCQAWDSSVVFGGGTQLIILAAA
>scFv VH
(SEQ ID NO: 19)
QVQLQQSGPGLVKPSQTLSLTCAISGDSVSSNSAAWNWIRQSPSRGLEWLGRAYYRSTWY
NDYAASVKSRITINPDTSKNQFSLQLNSVTPEDTAVYYCARGVNWNDDAFDIWGQGTMVT
VSS
>scFv VL
(SEQ ID NO: 20)
AIQMTQSPSSLSASVGDRVTITCRASQSISSYLNWYQQKPGKAPKLLVYAASSLQSGVPS
RFSGSGSGTDFTLTISSLQPEDFATYYCQQSYSTPLTFGGGTKVEIK
>(EA₃K)₃ rigid linker
(SEQ ID NO: 21)
EAAAKEAAAKEAAAK
>Full length open reading frame containing
(G₄S)₃ flexible linker
(SEQ ID NO: 22)
METDTLLLWVLLLWVPGSTGQVQLVQSGAEVKKPGESLKISCKGSRHSFTSYWIGWVRQM
PGKGLEWMGIIYPGDSDTRYSPSFQGQVTISADKSISTAYLQWSSLKASDTAMYYCARRI
AAAGIGNEYFDYWGQGTTVTVSSGGGGSGGGGSGGGGSSYELTQPPSVSVSPGQTASITC
SGDKLGDKYVSWYQQKPGQSPVLVIYQDSKRPSGIPERFSGSNSGNTATLTISGTQAMDE
ADYYCQAWDSSVVFGGGTQLIILAAAGGGGSGGGGSGGGGSQVQLQQSGPGLVKPSQTLS
LTCAISGDSVSSNSAAWNWIRQSPSRGLEWLGRAYYRSTWYNDYAASVKSRITINPDTSK
NQFSLQLNSVTPEDTAVYYCARGVNWNDDAFDIWGQGTMVTVSSGGGGSGGGGSGGGGSA
IQMTQSPSSLSASVGDRVTITCRASQSISSYLNWYQQKPGKAPKLLVYAASSLQSGVPSR
FSGSGSGTDFTLTISSLQPEDFATYYCQQSYSTPLTFGGGTKVEIKAAATTTPAPRPPTP
APTIASQPLSLRPEACRPAAGGAVHTRGLDFACDIYIWAPLAGTCGVLLLSLVITLYCKR
GRKKLLYIFKQPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEAYSEIGMKGER
RRGKGHDGLYQGLSTATKDTYDALHMQALPPRLE
>Full length open reading frame containing
(EA₃K)₃ rigid linker
(SEQ ID NO: 23)
METDTLLLWVLLLWVPGSTGQVQLVQSGAEVKKPGESLKISCKGSRHSFTSYWIGWVRQM
PGKGLEWMGIIYPGDSDTRYSPSFQGQVTISADKSISTAYLQWSSLKASDTAMYYCARRI
AAAGIGNEYFDYWGQGTTVTVSSGGGGSGGGGSGGGGSSYELTQPPSVSVSPGQTASITC
SGDKLGDKYVSWYQQKPGQSPVLVIYQDSKRPSGIPERFSGSNSGNTATLTISGTQAMDE
ADYYCQAWDSSVVFGGGTQLIILAAAEAAAKEAAAKEAAAKQVQLQQSGPGLVKPSQTLS
LTCAISGDSVSSNSAAWNWIRQSPSRGLEWLGRAYYRSTWYNDYAASVKSRITINPDTSK
NQFSLQLNSVTPEDTAVYYCARGVNWNDDAFDIWGQGTMVTVSSGGGGSGGGGSGGGGSA
IQMTQSPSSLSASVGDRVTITCRASQSISSYLNWYQQKPGKAPKLLVYAASSLQSGVPSR
FSGSGSGTDFTLTISSLOPEDFATYYCQQSYSTPLTFGGGTKVEIKAAATTTPAPRPPTP
APTIASQPLSLRPEACRPAAGGAVHTRGLDFACDIYIWAPLAGTCGVLLLSLVITLYCKR
GRKKLLYIFKQPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEAYSEIGMKGER
RRGKGHDGLYQGLSTATKDTYDALHMQALPPRLE

What is claimed:

1. A chimeric DNA comprising: a first DNA segment encoding a single-chain variable domain comprising an antibody heavy chain variable region (VH) (SEQ ID NO: 2) linked to an antibody light chain variable region (VL) (SEQ ID NO: 3) by a flexible linker (SEQ ID NO: 7) or a first DNA segment encoding a single-chain variable domain comprising a VH (SEQ ID NO: 5) linked to a VL (SEQ ID NO: 6) by a flexible linker (SEQ ID NO: 7), or a first DNA segment encoding a single-chain variable domain comprising a VH (SEQ ID NO: 12) linked by a flexible linker (SEQ ID NO: 7) to a VL (SEQ ID NO: 13) linked by a flexible linker (SEQ ID NO: 7) to a second DNA segment encoding a single-chain variable domain comprising a VH (SEQ ID NO: 14) linked to a VL (SEQ ID NO: 15) by a flexible linker (SEQ ID NO: 7), or a first DNA segment encoding a single-chain variable domain comprising a VH (SEQ ID NO: 17) linked by a flexible linker (SEQ ID NO: 7) to a VL (SEQ ID NO: 18) linked by a rigid linker (SEQ ID NO: 21) to a second DNA segment encoding a single-chain variable domain comprising a VH (SEQ ID NO: 19) linked to a VL (SEQ ID NO: 20) by a flexible linker (SEQ ID NO: 7) and a second DNA encoding partially or entirely an extracellular domain comprising at least a portion of the CD8 alpha extracellular domain (SEQ ID NO: 8), a transmembrane domain and intracellular domain of an endogenous protein and optionally stimulatory (SEQ ID NO: 10) and co-stimulatory (SEQ ID NO: 9) domains of an endogenous protein.

2. The chimeric DNA according to claim 1, wherein the endogenous protein is expressed on the surface of a lymphocyte or macrophage and triggers the activation of the lymphocyte or macrophage.

3. The chimeric DNA according to claim 2, wherein the chimeric DNA, upon transfection or transduction to the lymphocyte or macrophage, expresses both the single-chain variable domain and the domains of the endogenous protein in one single, continuous chain on the surface of the transfected or transduced lymphocytes or macrophages such that the transfected or transduced lymphocyte or macrophage is triggered to activate and/or proliferate and have MHC non-restricted antibody-type specificity when the expressed single chain variable domain binds to its antigen.

4. A chimeric DNA according to claim 1, wherein the second DNA segment further comprises partially or entirely the extracellular domain of the endogenous protein.

5. A chimeric DNA according to claim 1, wherein the second DNA segment further comprises partially or entirely the transmembrane domain of the endogenous protein.

6. A chimeric DNA according to claim 1, wherein the second DNA segment further comprises partially or entirely the co-stimulatory domain of the endogenous protein.

7. A chimeric DNA according to claim 1, wherein the second DNA segment comprises either entirely or partially domains of the CD28 family of receptors.

8. A chimeric DNA according to claim 1, wherein the second DNA segment comprises either entirely or partially domains of CD28 or ICOS.

9. A chimeric DNA according to claim 1, wherein the second DNA segment comprises entirely or partially domains of the TNFR superfamily.

10. A chimeric DNA according to claim 1, wherein the second DNA segment comprises entirely or partially domains of CD137, CD30, CD40, OX40, CD27, CD226, GITR, RANK, TACI, BCMA and BAFF-R.

11. A chimeric DNA according to claim 1, wherein the second DNA segment further comprises partially or entirely a lymphocyte receptor chain.

12. A chimeric DNA according to claim 1, wherein the second DNA segment comprises either entirely or partially a chain of the T-cell receptor.

13. A chimeric DNA according to claim 1, wherein the second DNA segment encodes the ζ chain of the T-cell receptor.

14. An expression vector comprising a chimeric DNA according to claim 1.

15. A lymphocyte or macrophage, isolated from an individual, transformed with an expression vector according to claim 14, wherein expression of the chimeric DNA endows the lymphocyte or macrophage with antibody specificity.

16. A lymphocyte or macrophage, isolated from an individual, transformed with a chimeric DNA according to claim 1, wherein expression of the chimeric DNA endows the lymphocyte or macrophage with antibody specificity.

17. A lymphocyte or macrophage according to claim 16, selected from the group consisting of a natural killer cell, a natural killer T-cell, a lymphokine activated killer cell, a cytotoxic T-cell, a helper T-cell, a gamma delta T-cell and a subtype thereof.

18. A chimeric DNA according to claim 1, wherein the first DNA segment encodes the single-chain variable domain of an antibody targeting a protein expressed or overexpressed on tumor cells.

19. A chimeric DNA according to claim 1, wherein the first DNA segment encodes the single-chain variable domain of an antibody targeting tumor cells either expressing or overexpressing CD19 or CD22, or a combination thereof.

20. A chimeric DNA according to claim 1 comprising SEQ ID NO: 1.

21. A chimeric DNA according to claim 1 comprising SEQ ID NO: 4.

22. A chimeric DNA according to claim 1 comprising SEQ ID NO: 11.

23. A chimeric DNA according to claim 1 comprising SEQ ID NO: 16.

24. A chimeric DNA according to claim 1 comprising SEQ ID NO: 22.

25. A chimeric DNA according to claim 1 comprising SEQ ID NO: 23.
